(19) United States
(12) Patent Application Publication
(10) Pub. No.: US 2023/0404904 A1
(43) Pub. Date: Dec. 21, 2023

(54) PET FOOD COMPOSITIONS

(71) Applicant: Hill's Pet Nutrition, Inc., Topeka, KS (US)

(72) Inventors: Dayakar BADRI, Lawrence, KS (US); Matthew JACKSON, Topeka, KS (US); Dale SCHERL, Lawrence, KS (US)

(73) Assignee: Hill's Pet Nutrition, Inc., Topeka, KS (US)

(21) Appl. No.: 18/335,208

(22) Filed: Jun. 15, 2023

Related U.S. Application Data
(60) Provisional application No. 63/352,434, filed on Jun. 15, 2022.

(51) Int. Cl. A61K 8/9789; A23K 50/40; A23K 20/158; A23K 20/147; A23K 20/163; A23K 20/26; A23K 10/37; A23K 20/121; A61K 8/37; A61Q 11/00

(52) U.S. Cl. CPC A61K 8/9789; A23K 50/40; A23K 20/158; A23K 20/147; A23K 20/163; A23K 20/26; A23K 10/37; A23K 20/121; A61K 8/375; A61Q 11/00; A61K 2800/28

(57) ABSTRACT

Pet food compositions and methods for minimizing tartar and/or improving the health of pets are disclosed herein. The pet food compositions typically include about 20 wt. % or more of fat comprising a medium chain triglyceride(s) having an octanoate component, wherein the medium chain triglyceride(s) comprising the octanoate component comprises about 10 wt. % or more of the fat, based on the total weight of the fat on a dry matter basis. The pet food compositions also may have a ketogenic ratio of about 1.5 or greater as calculated using Formula 1: (0.9 F+0.46 P)/(0.1 F+0.58 P+C), wherein F is the weight percentage of fat, P is the weight percentage of protein, and C is the weight percentage of carbohydrate.

### CROSS-REFERENCE TO RELATED PATENT APPLICATIONS

This application claims priority to U.S. Appl. No. 63/352,434, titled PET FOOD COMPOSITIONS and filed Jun. 15, 2022, the content of which is incorporated herein by reference in its entirety for all purposes.

### BACKGROUND

Many pets suffer from dental health problems. It is estimated that over 70% of dogs have some degree of gingival or periodontal disease. Plaque formation on the surface of the pets' teeth is a primary factor in the development of such problems. Dental plaque is a combination of bacteria, bacterial by-products that form a glycoprotein matrix, and salivary precipitates which bind to the tooth surface.

Contributing to the problem is the formation of tartar or dental calculus (mineralized plaque). Dental calculus forms on the tooth surface and serves as a substrate for the additional accumulation of plaque. Apart from causing gum irritation and potentially, periodontal disease if left untreated, has an unsightly appearance.

Pets are more susceptible to the formation of plaque and calculus than animals in the wild. This is due to the nature of the foods that pets eat. In the wild, many animals eat foods that mechanically abrade plaque and calculus from the teeth. In contrast, pets are usually fed pet foods that generally provide better nutritional value, but do not in general subject the teeth to abrasive forces sufficient to clean the teeth.

Maintenance of optimal dental health depends on regular professional cleaning of all these accumulations both above and below the gum line, as well as adequate home care. Mechanical cleansing of the tooth surfaces with tooth brushing is an effective means of controlling the buildup of plaque and calculus in both humans and pets. However, most pet owners are reluctant or unable to provide the routine brushing necessary to maintain good oral health in their pets.

There have been various attempts to provide products for improving pet dental health. One such attempt centers around the use of chews made from rawhide or rawhide substitutes. By allowing the pet to gnaw or chew on such rawhide products, the pet abrades calculus and plaque from its teeth. It is also possible, as described in European patent application 0272968, to incorporate various oral care agents into such chews. These products, however, have the drawback that they are typically expensive. Moreover, they are usually only effective with dogs that are prone to chew and gnaw.

Thus, there has been development and research into pet food compositions that when chewed by pets, cause a mechanical, abrasive cleaning of the pets' teeth.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to pet food compositions and methods of use thereof for reducing plaque and tartar accumulation and/or improving the oral health of pets. Additionally, the inventors discovered that certain pet food compositions can advantageously provide health benefits associated with ketogenic diets in addition to reductions of plaque and tartar accumulation. In accordance with one aspect, provided is a pet food composition comprising about 20 wt. % or more of fat, wherein the fat comprises one or more medium chain triglyceride having an octanoate component. The one or more medium chain triglyceride comprising the octanoate component comprises about 10 wt. % or more of the fat, based on the total weight of the fat on a dry matter basis. Additionally, the pet food composition includes a protein and a fiber, wherein the pet food composition comprises about 10 wt. % or less of carbohydrate and about 5 wt. % or less of digestible starch. Further, the pet food composition is typically formulated to have a ketogenic ratio of about 1.5 or greater as calculated according to Formula 1: (0.9 F+0.46 P)/(0.1 F+0.58 P+C), wherein F is the weight percentage of fat, P is the weight percentage of protein, and C is the weight percentage of carbohydrate, and all weight percentages are based on the total weight of the pet food composition on a dry matter basis.

According to another aspect, provided is a pet food composition comprising about 20 to about 60 wt. % of fat, wherein the fat comprises one or more medium chain triglyceride having an octanoate component. The one or more medium chain triglyceride comprising the octanoate component comprises about 10 wt. % or more of the fat. The pet food composition further includes from about 15 to about 55 wt. % of protein, from about 10 to about 60 wt. % of a fiber, and one or more fatty acid, wherein the pet food composition comprises about 10 wt. % or less of carbohydrate and about 5 wt. % or less of digestible starch, and wherein all weight percentages are based on the total weight of the pet food composition on a dry matter basis.

In accordance with yet a further aspect, a method is provided for reducing the accumulation of tartar comprising administering to a companion animal a pet food composition disclosed herein. The method may include providing and/or feeding the canine a pet food composition disclosed herein. In some instances, the method may include providing and/or feeding the canine the pet food compositions for 1 or more days, preferably 5 or more days, preferably 7 or more days, preferably 10 or more days, preferably 14 or more days, preferably 30 or more days, or preferably 42 or more days.

### BRIEF DESCRIPTION OF THE DRAWINGS

The features, and advantages of the invention will be apparent from the following more detailed description of certain embodiments of the invention and as illustrated in the accompanying drawings in which:

FIGS. 1 and 2 are bar graphs of the abundance levels of orange complex bacterial genera in the sub-gingival plaque of canines receiving non-limiting example pet food compositions and comparative pet food compositions in accordance with aspects of the invention.

It should be understood that the various aspects are not limited to the compositions, arrangements, and instrumentality shown in the figures.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not necessarily limited in its application to the details of any particular embodiment disclosed. The terminology used herein is for the purpose of description and not of limitation.

As used herein and in the appended claims, the singular forms “a”, “an”, and “the” include plural references unless the context dictates otherwise. The singular form of any class of the ingredients refers not only to one ingredient within that class, but also to a mixture of those ingredients. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the pet food composition. The symbol “°” refers to a degree, such as a temperature degree or a degree of an angle. The symbols “h”, “min”, “mL”, “nm”, “μm” means hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the pet food compositions of the instant disclosure can be free or essentially free of all components and elements positively recited throughout the instant disclosure. In some instances, the pet food compositions of the present disclosure may be substantially free of non-incidental amounts of the ingredient(s) or compound(s) described herein. A non-incidental amount of an ingredient or compound is the amount of that ingredient or compound that is added into the pet food composition by itself. For example, a pet food composition may be substantially free of a non-incidental amount of an ingredient or compound, although such ingredient(s) or compound(s) may be present as part of a raw material that is included as a blend of two or more compounds. Substantially free, unless other defined or described herein, typically refers to an ingredient or compound in an amount of about 2 wt. % or less, about 1.5 wt. % or less, about 1 wt. % or less, about 0.5 wt. % or less, about 0.1 wt. % or less, or about 0.05 wt. % or less, or about 0.01 wt. % or less, based on the total weight of the pet food composition on a dry matter basis.

Some of the various categories of components identified may overlap. In such cases where overlap may exist and the pet food composition includes both components (or the composition includes more than two components that overlap), an overlapping compound does not represent more than one component. For example, certain components or ingredients may be characterized as both an ancient grain and an amaranth. If a particular pet food care composition recites both an ancient grain and an amaranth, a compound that may be characterized as both an ancient and an amaranth will serve only as either an ancient or an amaranth—not both.

As used herein, the term “pet” could be used interchangeably with “companion animal” and refers to an animal of any species kept by a caregiver as a pet or any animal of a variety of species that have been widely domesticated as pets, including canines (Canis familiaris) and felines (Felis domesticus). Thus, a pet may include but is not limited to, working dogs, pet dogs, cats kept for rodent control (i.e. farm cats), pet cats, ferrets, birds, reptiles, rabbits, and fish.

To the extent that food and food ingredient contain water/moisture, the dry matter represents everything in the sample other than water including, for example, protein, fiber, fat, carbohydrates, minerals, etc. Dry matter weight is the total weight minus the weight of any water. The skilled artisan would readily recognize and understand nutritional amounts and percentages expressed as dry matter amounts, dry matter weights and dry matter percentages.

Dry matter intake per day is calculated as the total nutritional intake per day excluding all water. For example, an amount of an ingredient equal to a specific percent of daily nutritional intake refers to the amount of that ingredient in dry matter form (i.e., excluding all water) relative to the total amount of dry matter consumed (also excluding all water) in a day.

“Daily nutritional intake” and “total nutritional intake per day” refer to dry matter intake per day. That is, water weight is not included in calculating the amount of nutrition consumed per day. To calculate percent of an ingredient of total daily intake on a dry matter basis, water is removed from the total intake to give total daily dry matter intake and the percentage of the ingredient is based on amount of ingredient present as dry matter.

As used herein, an “ingredient” refers to any component of a pet food composition. The term “nutrient” refers to a substance that provides nourishment and thus has a nutrient value. In some cases, an ingredient may comprise more than one “nutrient,” for example, a composition may comprise corn comprising important nutrients including both protein and carbohydrate.

Aspects of the invention are directed to pet food compositions and methods of use thereof for reducing plaque and tartar accumulation and/or improving the oral health of pets. The inventors discovered that certain pet food compositions can also advantageously provide health benefits often associated with ketogenic diets in addition to reductions of tartar accumulation. For instance, certain embodiments disclosed herein may be used in methods or as a part of a diet for alleviating, mitigating, and/or reducing the symptoms of pets suffering from indications or conditions, including, but not limited to, cancer, inflammatory bowel disease, diabetes, obesity, hyperglycemia, hypertriglyceridemia, chronic inflammation, antibiotic resistance of the gut microbiome, or pathogenic dysbiosis of the gut microbiome, neurological seizures, high blood glucose, metabolic stress, or combinations thereof.

Preferably, the pet food compositions are formulated to have a ketogenic ratio of about 1.5 or greater as calculated according to Formula 1: (0.9 F+0.46 P)/(0.1 F+0.58 P+C), wherein F is the weight percentage of fat, P is the weight percentage of protein, and C is the weight percentage of carbohydrate, and all weight percentages are based on the total weight of the pet food composition on a dry matter basis. In some embodiments, the pet food composition has ketogenic ratio as calculated by Formula 1 of about 1.75 or greater, about 2 or greater, about 2.25 or greater, about 2.5 or greater, about 2.75 or greater, about 3 or greater, about 3.5 or greater, about 4 or greater, or about 5 or greater. Although the pet food composition may be formulated to have a ketogenic ratio as calculated according to Formula 1 of more than 5, in some embodiments, the pet food composition has a ketogenic ratio as calculated by Formula 1 of about 1.5 to about 4, about 1.7 to about 4, about 2 to about 4, or about 2.5 to about 4. Further discussion of pet food compositions having formulations that may have a desirable ketogenic ratio as calculated according to Formula 1 can be found in U.S. patent application Ser. No. 17/250,440, which is incorporated herein by reference in its entirety for all purposes.

In some embodiments, the pet food composition is formulated to comprises less than about 2 wt. % of phosphorus, based on the total weight of the pet food composition on a dry matter basis. For instance, the pet food composition may comprise less than about 1.5 wt. %, less than about 1 wt. %, less than about 0.7 wt. %, less than about 0.5 wt. %, less than about 0.1 wt. %, based on the total weight of the pet food composition on a dry matter basis. In at least one embodiment, the pet food composition is substantially free of or free of phosphorous.

Suitable components, such as those listed below, may be included or excluded from the formulations for the pet food compositions depending on the specific combination of other ingredients and the form of the pet food compositions. In some embodiments, the pet food compositions disclosed herein may be in the form of a stand-alone pet food, as a supplement to pet food, as a pet food treat, or the like.

The pet food composition are formulated to include fat in an amount typically ranging from about 20 wt. % or more, based on the total weight of the pet food composition on a dry matter basis. For example, the amount of fat present in the pet food composition may be in a range from about to about 60 wt. %, about 20 to about 55 wt. %, about 20 to about 50 wt. %, about 20 to about 45 wt. %, about 20 to about 40 wt. %, about 20 to about 35 wt. %, about 20 to about 30 wt. %; from about 23 to about 60 wt. %, about 23 to about 55 wt. %, about 23 to about 50 wt. %, about 23 to about 45 wt. %, about 23 to about 40 wt. %, about 23 to about 35 wt. %; from about 26 to about 60 wt. %, about 26 to about 55 wt. %, about 26 to about 50 wt. %, about 26 to about 45 wt. %, about 26 to about 40 wt. %, about 26 to about 35 wt. %; from about 29 to about 60 wt. %, about 29 to about wt. %, about 29 to about 50 wt. %, about 29 to about 45 wt. %, about 29 to about 40 wt. %, about 29 to about 35 wt. %; from about 32 to about 60 wt. %, about 32 to about 55 wt. %, about 32 to about 50 wt. %, about 32 to about 45 wt. %, about 32 to about 40 wt. %, based on the total weight of the pet food composition on a dry matter basis.

The term “fat” generally refers to a lipid or mixture of lipids that may generally be a solid or a liquid at ordinary room temperatures (e.g., 25° C.) and pressures (e.g., 1 atm). In some instances, the fat may be a viscous liquid or an amorphous solid at standard room temperature and pressure.

The fat in the pet food composition typically comprises one or more medium chain triglyceride(s), with at least one medium chain triglyceride having an octanoate component. Additionally, the pet food compositions are generally formulated such that the medium chain triglyceride(s) having an octanoate component comprises about 5 wt. % or more of the fat in the pet food composition. For example, the total amount of fat in the pet food composition may be comprised of medium chain triglyceride(s) having an octanoate in an amount of about 5 to about wt. %, about 5 to about 50 wt. %, about 5 to about 45 wt. %, about 5 to about 40 wt. %, about 5 to about 37 wt. %, about 5 to about 34 wt. %, about 5 to about 31 wt. %, about 5 to about 28 wt. %, about 5 to about 25 wt. %, about 5 to about 22 wt. %, about 5 to about 17 wt. %, about 5 to about 12 wt. %; from about 12 to about 60 wt. %, about 12 to about 50 wt. %, about 12 to about 45 wt. %, about 12 to about 40 wt. %, about 12 to about 37 wt. %, about 12 to about 34 wt. %, about 12 to about 31 wt. %, about 12 to about 28 wt. %, about 12 to about 25 wt. %, about 12 to about 22 wt. %, about 12 to about 17 wt. %; from about 17 to about 60 wt. %, about 17 to about 50 wt. %, about 17 to about 45 wt. %, about 17 to about 40 wt. %, about 17 to about 37 wt. %, about 17 to about 34 wt. %, about 17 to about 31 wt. %, about 17 to about 28 wt. %, about 17 to about 25 wt. %; from about 23 to about 60 wt. %, about 23 to about 50 wt. %, about 23 to about 45 wt. %, about 23 to about 40 wt. %, about 23 to about 37 wt. %, about 23 to about 34 wt. %, about 23 to about 31 wt. %; from about 27 to about 60 wt. %, about 27 to about 50 wt. %, about 27 to about 45 wt. %, about 27 to about 40 wt. %, about 27 to about 37 wt. %, about 27 to about 34 wt. %, about 27 to about 31 wt. %; from about 31 to about 60 wt. %, about 31 to about 50 wt. %, about 31 to about 45 wt. %, about 31 to about 40 wt. %, about 31 to about 37 wt. %; from about 35 to about 60 wt. %, about 35 to about 50 wt. %, about 35 to about 45 wt. %, about 35 to about 40 wt. %; from about 39 to about wt. %, about 39 to about 50 wt. %, about 39 to about 45 wt. %; from about 43 to about 60 wt. %, about 43 to about 50 wt. %, about 47 to about 60 wt. %, or any range or subrange thereof, based on the total weight of the fat in the pet food composition on a dry matter basis.

In addition to the at least one medium chain triglyceride having alphatic chains comprising an octanoate component, the fat may comprise one or more additional triglyceride(s). The additional triglyceride(s) may have one, two, or all three aliphatic chain components selected from pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, and mixtures thereof. In at least one preferred embodiment, the additional triglyceride(s) is selected from medium chain triglycerides.

As used herein, “medium chain triglycerides” refers to a triglyceride having at least one aliphatic carbon chain comprised of 6 to 10 carbons. For example, a medium chain triglyceride may have one, two, or three aliphatic carbons chains of 6 to 10 carbons. In some embodiments, the additional medium chain triglyceride has an aliphatic carbon chain of 6 carbons, 7 carbons, 8 carbons, 9 carbons, and/or 10 carbons. For example, the aliphatic carbon chain of the medium chain triglyceride may include from 6 to 9 carbons, 6 to 8 carbons, 6 or 7 carbons; 7 to 10 carbons, 7 to 9 carbons, 7 or 8 carbons; 8 to 10 carbons, or 8 or 9 carbons, or 9 or 10 carbons. In some embodiments, the medium chain triglyceride(s) comprises aliphatic carbon chains having 6 carbons, 8 carbons, or 10 carbons. For example, the additional medium chain triglyceride may have aliphatic carbon chains selected from octanoate and/or decanoate.

The pet food composition may comprise two, three, four, five, six, seven or more triglycerides, all of which may be selected from medium chain triglycerides. The pet food composition may in some preferred embodiments be formulated such that at least 50 wt. % of the total amount of medium chain triglycerides is medium chain triglycerides having an octanoate component, based on the total weight of medium chain triglycerides. For example, the medium chain triglycerides may be comprised of medium chain triglycerides having an octanoate component in an amount from about 50 up to 100 wt. %, about 55 up to 100 wt. %, about 60 up to 100 wt. %, about 65 up to 100 wt. %, about 70 up to 100 wt. %, about 75 up to 100 wt. %, about 80 up to 100 wt. %, about 85 up to 100 wt. %, about 90 up to 100 wt. %, about 95 up to 100 wt. %, about 97 up to 100 wt. %, about 98 up to 100 wt. %, about 99 up to 100 wt. %; from about 50 to about 95 wt. %, about 55 to about 95 wt. %, about 60 to about 95 wt. %, about 65 to about 95 wt. %, about 70 to about 95 wt. %, about 75 to about 95 wt. %, about 80 to about 95 wt. %, about 85 to about 95 wt. %, about 90 to about 95 wt. %; from about 50 to about 90 wt. %, about 55 to about 90 wt. %, about 60 to about 90 wt. %, about 65 to about 90 wt. %, about 70 to about 90 wt. %, about 75 to about 90 wt. %, about 80 to about 90 wt. %, about 85 to about 90 wt. %; from about 50 to about 85 wt. %, about 55 to about 85 wt. %, about 60 to about 85 wt. %, about 65 to about 85 wt. %, about 70 to about 85 wt. %, about 75 to about 85 wt. %, about 80 to about 85 wt. %; from about 50 to about wt. %, about 55 to about 80 wt. %, about 60 to about 80 wt. %, about 65 to about 80 wt. %, about to about 80 wt. %, about 75 to about 80 wt. %; from about 50 to about 75 wt. %, about 55 to about 75 wt. %, about 60 to about 75 wt. %, about 65 to about 75 wt. %, about 70 to about 75 wt. %; from about 50 to about 70 wt. %, about 55 to about 70 wt. %, about 60 to about 70 wt. %, about 65 to about 70 wt. %; from about 50 to about 65 wt. %, about 55 to about 65 wt. %, about 60 to about wt. %; from about 50 to about 60 wt. %, about 55 to about 60 wt. %, based on the total weight of the medium chain triglycerides. In at least one embodiment, the one or more medium chain triglyceride consists essentially of or consists of medium chain triglyceride having an octanoate component.

The pet food composition may, in some case, include a medium chain triglyceride having an aliphatic chain comprising a decanoate component. For example, the fat of the pet food composition may comprise a medium chain triglyceride having aliphatic chains comprising an octanoate component and a medium chain triglyceride having aliphatic chains comprising a decanoate component.

The inventors recognized that medium chain triglycerides having an octanoate (C8) component are more ketogenic than medium chain triglycerides having a decanoate (C10) component or dodecanoate (C12) component. Without being limited to any particular theory, it is believed that pet food compositions having an enhanced amount of medium chain triglycerides having an octanoate component and, preferably a reduced amount of medium chain triglycerides having a deconoate component can yield metabolic benefits achieved under ketotsis. In certain preferred embodiments, the pet food compositions is formulated to control the amount of medium chain triglycerides having aliphatic carbon chains of 10 to 12 carbons (e.g., a decanoate (C10) component). For example, the pet food composition may be adapted such that the fat comprises medium chain triglycerides having aliphatic carbon chains of 10 to 12 carbons (such as, a decanoate component or dodecanoate component) in an amount of about 4 wt. % or less, e.g., about 3 wt. % or less, about 2 wt. % or less, about 1 wt. % or less, about 0.5 wt. % or less, about 0.1 wt. % or less, about 0.05 wt. % or less, about 0.02 wt. % or less, or about 0.01 wt. % or less, based on the total weight of the fat in the pet food composition on a dry matter basis. In at least one embodiment, the pet food composition may be essentially free of or free of medium chain triglycerides having aliphatic carbon chains of 10 to 12 carbons and/or a decanoate component.

Additionally or alternatively, the pet food composition may be formulated to have a weight ratio of the medium chain triglyceride having aliphatic chains comprising an octanoate component to the medium chain triglyceride having aliphatic chains comprising an decanoate component of from about 200:1 to about 500:1. In certain embodiments, the weight ratio of the medium chain triglyceride(s) having aliphatic chains comprising an octanoate component to the medium chain triglyceride(s) having aliphatic chains comprising a decanoate component is from about 250:1 to about 500:1, e.g., from about 300:1 to about 500:1, about 350:1 to about 500:1, about 400:1 to about 500:1; from about 250:1 to about 450:1, about 300:1 to about 450:1, about 350:1 to about 450:1, about 400:1 to about 450:1; from about 250:1 to about 400:1, about 300:1 to about 400:1, about 350:1 to about 400:1, or any range or subrange thereof.

In some embodiments, however, the weight ratio of the medium chain triglyceride(s) having an aliphatic chain comprising an octanoate component to the medium chain triglyceride(s) having an aliphatic chain comprising an decanoate component is from about 2:1 to about 20:1, about 2:1 to about 17:1, about 2:1 to about 15:1, about 2:1 to about 13:1, about 2:1 to about 11:1, about 2:1 to about 9:1, about 2:1 to about 7:1; from about 4:1 to about 20:1, about 4:1 to about 17:1, about 4:1 to about 15:1, about 4:1 to about 13:1, about 4:1 to about 11:1, about 4:1 to about 9:1, about 4:1 to about 7:1; from about 5:1 to about 20:1, about 5:1 to about 17:1, about 5:1 to about 15:1, about 5:1 to about 13:1, about 5:1 to about 11:1, about 5:1 to about 9:1, about 5:1 to about 7:1; from about 6:1 to about 20:1, about 6:1 to about 17:1, about 6:1 to about 15:1, about 6:1 to about 13:1, about 6:1 to about 11:1, about 6:1 to about 9:1, or any range or subrange thereof.

In some embodiments, the pet food compositions are formulated such that the total amount of medium chain triglyceride(s) comprises about 10 wt. % or more of the fat in the pet food composition. The total amount of medium chain triglycerides may comprise, e.g., medium chain triglycerides having an aliphatic chain comprising an octanoate component and any additional medium chain triglycerides, if present. For example, the total amount of fat in the pet food composition may be comprised of medium chain triglyceride(s) in a total amount of from about 10 to about 60 wt. %, about 10 to about 50 wt. %, about 10 to about 45 wt. %, about 10 to about 40 wt. %, about 10 to about 37 wt. %, about 10 to about 34 wt. %, about 10 to about 31 wt. %, about 10 to about 28 wt. %, about 10 to about 25 wt. %, about 10 to about 22 wt. %, about 10 to about 17 wt. %, about 10 to about 12 wt. %; from about 12 to about 60 wt. %, about 12 to about 50 wt. %, about 12 to about 45 wt. %, about 12 to about 40 wt. %, about 12 to about 37 wt. %, about 12 to about 34 wt. %, about 12 to about 31 wt. %, about 12 to about 28 wt. %, about 12 to about 25 wt. %, about 12 to about 22 wt. %, about 12 to about 17 wt. %; from about 17 to about 60 wt. %, about 17 to about 50 wt. %, about 17 to about 45 wt. %, about 17 to about 40 wt. %, about 17 to about 37 wt. %, about 17 to about 34 wt. %, about 17 to about 31 wt. %, about 17 to about 28 wt. %, about 17 to about 25 wt. %; from about 23 to about 60 wt. %, about 23 to about 50 wt. %, about 23 to about 45 wt. %, about 23 to about 40 wt. %, about 23 to about 37 wt. %, about 23 to about 34 wt. %, about 23 to about 31 wt. %; from about 27 to about 60 wt. %, about 27 to about 50 wt. %, about 27 to about 45 wt. %, about 27 to about 40 wt. %, about 27 to about 37 wt. %, about 27 to about 34 wt. %, about 27 to about 31 wt. %; from about 31 to about 60 wt. %, about 31 to about 50 wt. %, about 31 to about 45 wt. %, about 31 to about 40 wt. %, about 31 to about 37 wt. %; from about 35 to about 60 wt. %, about 35 to about 50 wt. %, about 35 to about 45 wt. %, about 35 to about 40 wt. %; from about 39 to about 60 wt. %, about 39 to about 50 wt. %, about 39 to about 45 wt. %; from about 43 to about 60 wt. %, about 43 to about 50 wt. %, about 47 to about 60 wt. %, or any range or subrange thereof, based on the total weight of the fat in the pet food composition on a dry matter basis.

Additionally or alternatively, the one or more triglyceride(s) may include a polyunsaturated fatty acid component selected from omega-3 fatty acids, omega-6 fatty acids, or a combination of two or more thereof. Examples of omega-3 fatty acid components that may comprise the aliphatic chain of the additional triglyceride include those selected from linolenic acid, stearidonic acid, eicosatetraenoic acid, eicosapentaenoic acid, docosapentaenoic acid, and/or docosahexaenoic acid. Examples of omega-6 fatty acid components that may comprise the aliphatic chain of an additional triglyceride include linolenic acid, calendic acid, eicosadienoic acid, arachidonic acid, docosadienoic acid, adrenic acid, osbond acid, tetracosatetraenoic acid, and/or tetracosapentaenoic acid. In some embodiments, the polyunsaturated fatty acid component of the additional triglyceride is selected from linolenic acid, and/or arachidonic acid.

The pet food compositions may, optionally, include one or more fatty acid(s). The fatty acid(s) when present in the pet food composition is typically in an amount from about 0.1 to about 5 wt. %, based on the total weight of the pet food composition on a dry matter basis. For example, the fatty acid may present in an amount from about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the pet food composition.

The one or more fatty acid(s) may be selected from omega-3 fatty acids, omega-6 fatty acids, lauric acid, myristic acid, palmitic acid, palmitoleic acid, margaric acid, margaroleic acid, stearic acid, oleic acid, stearidonic acid, gadoleic acid, behenic acid, erucic acid, docosatetra acid, and a combination of two or more thereof. In at least one preferred embodiment, the one or more fatty acid comprises an omega-3 fatty acid, an omega-6 fatty acid, or a combination of two or more thereof. Examples of omega-3 fatty acids include linolenic acid, stearidonic acid, eicosatetraenoic acid, eicosapentaenoic acid, docosapentaenoic acid, and docosahexaenoic acid. Examples of omega-6 fatty acids include linoleic acid, calendic acid, eicosadienoic acid, arachidonic acid, docosadienoic acid, adrenic acid, osbond acid, tetracosatetraenoic acid, and tetracosapentaenoic acid.

The pet food composition, in some embodiments, comprises one or more omega-3 fatty acid and one or more omega-6 fatty acid. In some instance, the pet food composition is formulated to have a weight ratio of omega-3 fatty acid(s) to omega-6 fatty acid(s) of from about 0.5:1 to about 7:1, about 0.5:1 to about 3:1, about 0.5:1 to about 2.5:1, about 0.5:1 to about 2:1, about 0.5:1 to about 1.5:1, about 0.5:1 to about 1:1, about 0.6:1 to about 7:1, about 0.6:1 to about 3:1, about 0.6:1 to about 2.5:1, about 0.6:1 to about 2:1, about 0.6:1 to about 1.5:1, or about 0.6:1 to about 1:1.

The fat may be incorporated completely within the food composition, deposited on the outside of the pet food composition, or a mixture of the two methods. In some embodiments, the pet food compositions further include an effective amount of one or more substances selected from the group consisting of glucosamine, chondroitin, chondroitin sulfate, methylsulfonylmethane (“MSM”), creatine, antioxidants, Perna canaliculata, and mixtures thereof.

Fat can be supplied by any of a variety of sources known by those skilled in the art, including meat, meat by-products, canola oil, fish oil such as anchovy oil and menhaden oil, and plants. Meat fat sources include poultry fat, turkey fat, pork fat, lard, tallow, and beef fat. Plant fat sources include wheat, flaxseed, rye, barley, rice, sorghum, corn, oats, millet, wheat germ, corn germ, soybeans, peanuts, and cottonseed, as well as oils derived from these and other plant fat sources such as corn oil, soybean oil, cottonseed oil, palm oil, palm kernel oil, linseed oil, canola oil, rapeseed oil, and/or olestra.

In some cases, the fat in the pet food composition is a crude fat. Crude fat may be included into the compositions in the amounts disclosed above with respect of the total fat, such as from about 8 to about 50 wt. %, based on the total weight of the pet food composition on a dry matter basis. In some embodiments, the pet food composition comprises crude fat in an amount of about to about 40 wt. %, about 12 to about 35 wt. %, about 14 to about 30 wt. %, about 16 to about 24 wt. %, based on the total weight of the pet food composition on a dry matter basis. In some cases, it may be preferable that about 50 wt. % or more, about 60 wt. % or more, about 70 wt. % or more, about 80 wt. % or more, or about 90 wt. % or more of the total fat is obtained from an animal source. Alternatively, about 50 wt. % or more, about 60 wt. % or more, about 70 wt. % or more, about 80 wt. % or more, or about 90 wt. % or more of the total fat may be obtained from a plant source.

The pet food composition includes protein in an amount that may vary. For instance, the pet food composition may include protein in an amount from about 15 to about 55 wt. %, based on the total weight of the pet food composition on a dry matter basis. In some embodiments, the amount of protein present in the pet food composition is from about 15 to about 50 wt. %, about to about 45 wt. %, about 15 to about 40 wt. %, about 15 to about 35 wt. %, about 15 to about 30 wt. %; about 20 to about 55 wt. %, about 20 to about 50 wt. %, about 20 to about 45 wt. %, about 20 to about 40 wt. %, about 20 to about 35 wt. %, about 20 to about 30 wt. %; from about 23 to about wt. %, about 23 to about 50 wt. %, about 23 to about 45 wt. %, about 23 to about 40 wt. %, about 23 to about 35 wt. %; from about 26 to about 55 wt. %, about 26 to about 50 wt. %, about 26 to about 45 wt. %, about 26 to about 40 wt. %, about 26 to about 35 wt. %; from about 29 to about 55 wt. %, about 29 to about 50 wt. %, about 29 to about 45 wt. %, about 29 to about 40 wt. %, about 29 to about 35 wt. %; from about 32 to about 55 wt. %, about 32 to about 50 wt. %, about 32 to about wt. %, about 32 to about 40 wt. %; from about 35 to about 55 wt. %, about 35 to about 50 wt. %, about 35 to about 45 wt. %, about 35 to about 40 wt. %; from about 38 to about 55 wt. %, about 38 to about 50 wt. %, about 38 to about 45 wt. %; from about 41 to about 55 wt. %, about 41 to about wt. %; or from about 43 to about 55 wt. %, including ranges or subranges thereof, based on the total weight of the pet food composition on a dry matter basis.

The protein of the pet food composition may comprises one or more amino acids selected from Tryptophan, Taurine, Histidine, Carnitine, Carnosine, Alanine, Cysteine, Arginine, Methionine (including DL-methionine, and L-methionine), Tryptophan, Lysine, Asparagine, Aspartate (Aspartic acid), Phenylalanine, Valine, Threonine, Isoleucine, Histidine, Leucine, Glycine, Glutamine, Taurine, Tyrosine, Homocysteine, Ornithine, Citruline, Glutamate (Glutamic acid), Proline, and/or Serine, and a combination of two or more thereof. In some cases, the one or more amino acid(s) may comprise essential amino acids. Essential amino acids are amino acids that cannot be synthesized de novo, or in sufficient quantities by an organism and thus must be supplied in the diet. Essential amino acids vary from species to species, depending upon the organism's metabolism. For example, it is generally understood that the essential amino acids for dogs and cats (and humans) are phenylalanine, leucine, methionine, lysine, isoleucine, valine, threonine, tryptophan, histidine and arginine. In addition, taurine, while technically not an amino acid but a derivative of cysteine, is an essential nutrient for cats.

A portion of the protein in the composition may be digested (e.g., hydrolyzed) protein. For example, the composition may include an amount of protein, where about 40 wt. % or more, about wt. % or more, about 60 wt. % or more, about 70 wt. % or more, about 80 wt. % or more, about wt. % or more, about 95 wt. % or more, about 98 wt. % or more, or about 99 wt. % or more of the protein is digested (e.g., hydrolyzed) protein. In some embodiments, e.g., when the composition desirable promotes weight loss, the portion of protein that is digested (e.g., hydrolyzed) protein is about 60 wt. % or less, about 50 wt. % or less, about 40 wt. % or less, about 30 wt. % or less, about wt. % or less, or about 10 wt. % or less, based on the total amount of protein in the pet food composition on a dry matter basis. In further embodiment, the amount of protein that is digested (e.g., hydrolyzed) protein is about 10 to about 99 wt. %, about 10 to about 95 wt. %, about 10 to about 90 wt. %, about 10 to about 70 wt. %, about 10 to about 50 wt. %, about 10 to about 30 wt. %; about 30 to about 99 wt. %, about 30 to about 95 wt. %, about 30 to about 90 wt. %, about 30 to about 70 wt. %, about 30 to about 50 wt. %; about 50 to about 99 wt. %, about 50 to about 95 wt. %, about 50 to about 90 wt. %, about 50 to about 70 wt. %; or about 70 to about 99 wt. %, about 70 to about 95 wt. %, about 70 to about 90 wt. %, including ranges and subranges therein, based on the total amount of protein in the pet food composition on a dry matter basis.

Protein may be supplied by any of a variety of sources known by those of ordinary skill in the art including plant sources, animal sources, microbial sources or a combination of these. For example, animal sources may include meat, meat-by products, seafood, dairy, eggs, etc. Meats, for example, may include animal flesh such as poultry, fish, and mammals including cattle, pigs, sheep, goats, and the like. Meat by-products may include, for example, lungs, kidneys, brain, livers, stomachs and intestines. Plant protein includes, for example, soybean, cottonseed, and peanuts. Microbial sources may be used to synthesize amino acids (e.g., lysine, threonine, tryptophan, methionine) or intact protein such as protein from sources listed below.

Examples of protein or protein ingredients may comprise chicken meals, chicken, chicken by-product meals, lamb, lamb meals, turkey, turkey meals, beef, beef by-products, viscera, fish meal, enterals, kangaroo, white fish, venison, soybean meal, soy protein isolate, soy protein concentrate, corn gluten meal, corn protein concentrate, distillers dried grains, and/or distillers dried grain solubles and single-cell proteins, for example yeast, algae, and/or bacteria cultures.

The protein can be intact, completely hydrolyzed, or partially hydrolyzed. The protein content of foods may be determined by any number of methods known by those of skill in the art, for example, as published by the Association of Official Analytical Chemists in Official Methods of Analysis (“OMA”), method 988.05. The amount of protein in a composition disclosed herein may be determined based on the amount of nitrogen in the composition according to methods familiar to one of skill in the art.

The pet food compositions are typically formulated to include fiber in an amount from about 10 to about 60 wt. %, based on the total weight of the pet food composition on a dry matter basis. For instance, the amount of fiber present in the pet food composition may be from about 10 to about 55 wt. %, about 10 to about 50 wt. %, about 10 to about 45 wt. %, about 10 to about 40 wt. %, about 10 to about 35 wt. %, about 10 to about 30 wt. %; from about 15 to about 60 wt. %, about 15 to about 55 wt. %, about 15 to about 50 wt. %, about 15 to about 45 wt. %, about 15 to about 40 wt. %, about 15 to about 35 wt. %, about 15 to about 30 wt. %; from about 20 to about 60 wt. %, about 20 to about 55 wt. %, about 20 to about 50 wt. %, about 20 to about 45 wt. %, about 20 to about 40 wt. %, about 20 to about 35 wt. %, about 20 to about 30 wt. %; from about 25 to about 60 wt. %, about 25 to about 55 wt. %, about 25 to about 50 wt. %, about 25 to about 45 wt. %, about 25 to about 40 wt. %, about 25 to about 35 wt. %; from about 30 to about 60 wt. %, about 30 to about 55 wt. %, about 30 to about 50 wt. %, about 30 to about 45 wt. %, about 30 to about 40 wt. %; from about 35 to about 60 wt. %, about 35 to about 55 wt. %, about 35 to about 50 wt. %, about 35 to about 45 wt. %; from about 40 to about 60 wt. %, about 40 to about 55 wt. %, about 40 to about 50 wt. %; from about 45 to about 60 wt. %, about 45 to about 55 wt. %; from about 50 to about 60 wt. %, including ranges and subranges thereof, based on the total weight of the pet food composition on a dry matter basis.

The total amount of fiber present in the pet food composition generally comprises an amount of crude fiber and dietary fiber. The amount of crude fiber and/or dietary fiber may be present in the pet food compositions in any of the above amounts disclosed for the total amount of fiber. Crude fiber includes indigestible components contained in cell walls and cell contents of plants such as grains, e.g., hulls of grains such as rice, corn, and beans.

Dietary fiber refers to components of a plant that are resistant to digestion by an animal's digestive enzymes. Dietary fiber includes soluble fiber and insoluble fiber. Soluble fibers are resistant to digestion and absorption in the small intestine and undergo complete or partial fermentation in the large intestine, e.g., beet pulp, guar gum, chicory root, psyllium, pectin, blueberry, cranberry, squash, apples, oats, beans, citrus, barley, or peas. Insoluble fibers can be supplied by any of a variety of sources, including, for example, cellulose, whole-wheat products, wheat oat, corn bran, flax seed, grapes, celery, green beans, cauliflower, potato skins, fruit skins (e.g., pear skin), vegetable skins, peanut hulls, almond shell, walnut shell, pecan shell, citrus pulp, beet pulp, and soy fiber. In some embodiments, the dietary fiber may be chosen from pecan shell, citrus pulp, beet pulp, and a combination of two or more thereof. Crude fiber includes indigestible components contained in cell walls and cell contents of plants such as grains, for example, hulls of grains such as rice, corn, and beans.

In some preferred embodiments, the pet food compositions may include one or more sclereid. The one or more sclereids may be obtained from nut shells, such as pecan shells, almonds shells, walnut shells, etc.; from seeds, such as pea, bean, soybean seeds, etc.; and/or from fruit skins, such as pear skin, etc. In one embodiment, the pet food composition includes pecan shell particles comprising one or more sclereids. The sclereid(s) may have a microscopic structure and, preferably, serve as an abrasive. For instance, the sclereid(s) may be selected to provide mechanical disruption of debris, food, and/or bacteria on the teeth of a pet consuming a pet food composition disclosed herein, which contains such sclereid(s). In at least one embodiment, the sclereid(s) may be selected to include one or more polyphenols.

In some embodiments, the pet food composition has a weight ratio of insoluble fiber to soluble fiber from about 20:1 to about 8:1. For example, the pet food composition may have a weight ratio of insoluble fiber to soluble fiber from about 18:1 to about 8:1, about 16:1 to about 8:1, about 14:1 to about 8:1, about 12:1 to about 8:1, about 10:1 to about 8:1. The pet food composition may have a weight ratio of insoluble fiber to soluble fiber of about 11:1.

Additionally and/or alternatively, the fiber component of the pet food composition may comprise an acid detergent fiber and/or a neutral detergent fiber. In some instances, the pet food composition includes an acid detergent fiber and a neutral detergent fiber, e.g., in an amount that is from about 6 to about 20 wt. %, based on the total weight of the pet food composition on a dry matter basis. For example, the pet food composition may include one of an acid detergent fiber and/or a neutral detergent fiber in an amount from about 3 to about 15 wt. %, about 3 to about 12 wt. %, about 3 to about 10 wt. %, about 3 to about 8 wt. %, about 3 to about 6 wt. %, about 3 to about 5 wt. %; about 5 to about 15 wt. %, about 5 to about 12 wt. %, about 5 to about 10 wt. %, about 5 to about 8 wt. %, about 5 to about 6 wt. %; about 7 to about 15 wt. %, about 7 to about 12 wt. %, about 7 to about 10 wt. %, about 7 to about 8 wt. %; about 9 to about 15 wt. %, about 9 to about 12 wt. %, about 9 to about 10 wt. %; about 11 to about 15 wt. %, about 11 to about 13 wt. %, or about 12 to about 15 wt. %, including ranges and subranges thereof, based on the total weight of the pet food composition on a dry matter basis.

In some embodiments, the pet food composition has a weight ratio of the acid detergent fiber to the neutral detergent fiber of from about 3:1 to about 1:3. Preferably, the weight ratio of the acid detergent fiber to the neutral detergent fiber is from about 2:1 to about 1:2. In some instances, the weight ratio of the acid detergent fiber to the neutral detergent fiber is from about 1.75:1 to about 1:1.75, about 1.5:1 to about 1:1.5, about 1.25:1 to about 1:1.25. In at least one embodiment, the weight ratio of the acid detergent fiber to the neutral detergent fiber is about 1:1.

The pet food composition may further comprise ash. As described herein, ash consists of compounds that are not organic or water, generally produced by combustion of biological materials. The ash may be present in the pet food composition in an amount ranging from about 1 to about 10 wt. %, based on the total weight of the food composition on a dry weight basis, including all amounts and sub-ranges there-between. In some embodiment, the ash may be present in the food composition in an amount ranging from about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %; from about 3 to about 10 wt. %, about 3 to about 8 wt. %, about 3 to about 6 wt. %, about 3 to about 5 wt. %; from about 4 to about 10 wt. %, about 4 to about 8 wt. %, about 4 to about 6 wt. %; from about 5 to about 10 wt. %, about 5 to about 8 wt. %, or any range or subrange thereof, based on the total weight of the food composition on a dry weight basis.

The pet food composition preferably contains carbohydrates in an amount of about 10 wt. % or less, based on the total weight of the pet food composition on a dry matter basis. In some embodiments, the pet food compositions have an amount of carbohydrates of about 9 wt. % or less, about 8 wt. % or less, about 7 wt. % or less, about 6 wt. % or less, about 5 wt. % or less, about 4 wt. % or less, about 3 wt. % or less, about 2 wt. % or less, about 1 wt. % or less, or about 0.5 wt. % or less, based on the total weight of the pet food composition on a dry matter basis. The inventors discovered that it may be advantageous to, additionally or alternatively, formulate the pet food compositions to contain about 5 wt. % or less of digestible starch, based on the total weight of the pet food composition on a dry matter basis. For example, the amount of digestible starch in the pet food compositions may be about 4 wt. % or less, about 3 wt. % or less, about 2 wt. % or less, about 1 wt. % or less, or about 0.5 wt. % or less, based on the total weight of the pet food composition on a dry matter basis. In some embodiments, the pet food compositions may also contain about 5 wt. % or less, about 4 wt. % or less, about 3 wt. % or less, about 2 wt. % or less, about 1 wt. % or less, or about 0.5 wt. % or less of sugar, based on the total weight of the pet food composition on a dry matter basis.

The term “carbohydrate” as used herein includes polysaccharides (e.g., starches and dextrins) and sugars (e.g., sucrose, lactose, maltose, glucose, and fructose) that are metabolized for energy when hydrolyzed. Generally, carbohydrate percentage can be calculated as nitrogen free extract (“NFE”), which can be calculated as follows: NFE %=100%-(moisture %)-(protein %)-(fat %)-(ash %)-(crude fiber %). One skilled in the art could manipulate the texture of the final product by properly balancing carbohydrate sources. For example, short chain polysaccharides lend to be sticky and gluey, and longer chain polysaccharides are less sticky and gluey than the shorter chain; the desired texture of this hybrid food is achieved by longer chain polysaccharide and modified starches such as native or modified starches, cellulose and the like. The carbohydrate mixture may additionally comprise optional components such as added salt, spices, seasonings, vitamins, minerals, flavorants, colorants, and the like. The amount of the optional components is at least partially dependent on the nutritional requirements for different life stages of animals.

Carbohydrates can be supplied by any of a variety of sources known by those skilled in the art, including, but not limited to, oat fiber, cellulose, peanut hulls, beet pulp, parboiled rice, cornstarch, corn gluten meal, cereal, and sorghum. Grains supplying carbohydrates can include, but are not limited to, wheat, durum, semolina, corn, barley, and rice. In certain embodiments, the carbohydrate component comprises a mixture of one or more carbohydrate sources. Carbohydrates content of foods can be determined by any number of methods known by those of skill in the art.

The pet food compositions may include one or more phenols, e.g., in an amount from about to about 10 wt. %, based on the total weight of the pet food composition on a dry matter basis. In some instance, the amount of phenols present in the pet food composition is from about 0.01 to about 10 wt. %, about 0.01 to about 8 wt. %, about 0.01 to about 6 wt. %, about 0.01 to about 5 wt. %, about 0.01 to about 4 wt. %, about 0.01 to about 3 wt. %, about 0.01 to about 2 wt. %, about to about 1 wt. %, about 0.01 to about 0.5 wt. %, about 0.01 to about 0.1 wt. %; from about 0.05 to about 10 wt. %, about 0.05 to about 8 wt. %, about 0.05 to about 6 wt. %, about 0.05 to about 5 wt. %, about 0.05 to about 4 wt. %, about 0.05 to about 3 wt. %, about 0.05 to about 2 wt. %, about to about 1 wt. %, about 0.05 to about 0.5 wt. %, about 0.05 to about 0.1 wt. %; from about 0.1 to about 10 wt. %, about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %; from about 3 to about 10 wt. %, about 3 to about 8 wt. %, about 3 to about 6 wt. %, about 3 to about 5 wt. %; from about 4 to about 10 wt. %, about 4 to about 8 wt. %, about 4 to about 6 wt. %; from about 5 to about 10 wt. %, about 5 to about 8 wt. %, or any range or subrange thereof, based on the total weight of the food composition on a dry weight basis.

The one or phenols may phenol selected from flavonoids, lignans, metabolic enhancers, and a combination of two or more thereof. Examples of lignin that may be included in the pet food composition include coniferyl alcohol, sinapyl alcohol, paracoumaryl alcohol, and combinations of two or more thereof. Non-limiting examples of flavonoids include, but are not limited to, flavones, flavonols and flavonones, which reportedly protect the vascular system and act to reduce allergies, inflammation, viruses and tumors. Flavonols reportedly strengthen the intertwined strands of collagen protein. Anthocyanidins, a type of flavonol may scavenge free radicals. Catechins and gallic acids are a type of flavonoid that reportedly have chemoprotective properties. Isoflavonoes reportedly block enzymes that promote tumor growth. Glucosinolates and allyl sulfides, both types of thiol phytonutrients, reportedly possess antimutagenic and anticarcinogenic properties as well as immune and cardiovascular protection in humans. Indoles interact with vitamin C and may bind carcinogens and enzymes. Isoprenoids neutralize free radicals. Lipoic acid and ubiquinone may act as antioxidants to extend the effects of other antioxidants. Natural sources of the at least one flavonoid and/or their conjugates may be selected from the group consisting of black or green tea, capers, lovage, apples, onion, in particular red onion, red grapes, citrus fruit, tomato, broccoli, raspberry, bog whortleberry, lingonberry, cranberry, chokeberry, sweet rowan, rowanberry, sea buckthorn berry, crowberry, prickly pear cactus fruit, or combinations thereof.

The metabolic enhancer may be selected from lipoic acid, taurine, carnitine, betaine (trimethylglycine), coenzyme Q10, beta-alanine, pyrroloquinoline quinone (PQQ), creatine, and a combination of two or more thereof. In one embodiment, the metabolic enhancer is lipoic acid, carnitine, or a combination thereof. In further embodiments, the pet food composition may contain added carnitine between 0.01 and 0.1%, added taurine between 0.1 and 0.5%, added betaine between 0.25 and 1% and/or added lipoic acid between 0.005 and 0.05%. Due to species differences, lipoic acid is not included in companion animal feline formulations. The inventors recognized that for some embodiments enhanced benefits of the pet food composition can be achieved by increasing to allow increased fat metabolism, e.g., under a ketogenic diet. For instance, it is believed that ketogenic diet induces a metabolic burden for catabolism of dietary fat due to the large percentage of energy intake as fat and can changes in gene expression and enzyme activity that result in a metabolic shift toward fat metabolism and increased energy expenditure. In fact, it may be that a ketogenic diet is not maximally valuable unless sufficient ancillary factors are available to allow increased fat metabolism.

The inventors recognized that certain additives may be advantageously incorporated to reduce or obviate the potential acidic metabolic burden on the circulatory and urinary systems due to ketogenic diets. For example, additives, such as potassium citrate may be incorporated into the pet food compositions disclosed herein to reduce kidney-stone incidence or formation. Potassium citrate may provide benefits to urinary health including dissolution of uroliths. It is noted that the incorporation of potassium citrate requires a non-obvious balancing of various factors/variables including, but not limited to, cations and anions, residual potassium, the resultant urinary pH, overall palatability, or combinations thereof. Nevertheless, in some embodiments, the pet food composition may contain potassium citrate in an amount sufficient to preferably provide between 1 to 6 Milliequivalents (mEq) potassium citrate per kilogram metabolic bodyweight (1 to 6 mEq K3Citrate/kg0.75). The pet food composition may preferably provide between 2 to 4 mEq K3Citrate/kg0.75. The pet food composition may in some cases provide about 3 mEq K3Citrate/kg0.75.

In certain embodiments, the pet food composition comprises moisture. The moisture may be present at various amounts or concentrations. In one embodiment, moisture may be present in an amount of from about 3 to about 20 wt. %, based on the total weight of the pet food composition. For example, moisture may be present in an amount of about 3 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, about 10 wt. %, about 10.5 wt. %, about 11 wt. %, about 11.5 wt. %, about 12 wt. %, about 12.5 wt. %, about 13 wt. %, about 13.5 wt. %, about 14 wt. %, about 14.5 wt. %, or about 15 wt. %, based on the total weight of the pet food composition. In another example, moisture may be present in an amount of from about 6% to about 12%, about 9% to about 13%, about 9% to about 11%, or about 9% to about 13%, based on the total weight of the pet food composition. In certain embodiments, moisture is present in an amount of about 5% to about 12%, about 6% to about 11%, or about 7% to about 10.0%, based on the total weight of the pet food composition. In further embodiments, moisture is present in an amount of about 65% to about 85%, about 60% to about 80%, or about 60% to about 75%, based on the total weight of the pet food composition.

The pet food compositions may include one or more ingredients and/or sources of glucose mimetic, carotenoids, and/or arginine and derivatives thereof. Sources of glucose mimetics may comprise glucose anti-metabolites including 2-deoxy-D-glucose, 5-thio-D-glucose, 3-O-methylglucose, anhydrosugars including 1,5-anhydro-D-glucitol, 2,5-anhydro-D-glucitol, and 2,5-anhydro-D-mannitol, mannoheptulose, and/or avocado extract comprising mannoheptulose. Sources of carotenoids may include lutein, astaxanthin, zeaxanthin, bixin, lycopene, and/or beta-carotene. Sources of antioxidant ingredients may comprise tocopherols (vitamin E), vitamin C, vitamin A, plant-derived materials, carotenoids (described above), selenium, and/or CoQ10 (Co-enzyme Q10). In a preferred embodiment, the pet food composition contains high levels of arginine and derivatives thereof. The amount of arginine present in the composition may be about to about 10.0 wt. %, about 0.01 to about 5.0 wt. %, about 0.01 to about 2.0 wt. %, about 0.1 to about 10.0 wt. %, about 0.1 to about 5.0 wt. %, about 0.1 to about 2.0 wt. %; about 0.5 to about 5.0 wt. %, about 0.5 to about 2.0 wt. %, about 1.5 to about 5.0 wt. %, about 1.5 to about 2.0 wt. %, about 0.5 wt. %, about 1.0 wt. %, about 1.4 weight %, about 1.44 wt. %, about 1.8 wt. %, or about 2.0 wt. %, based on the total weight of the composition on a dry matter basis. The arginine present in the composition may be L-arginine, D-arginine, or a mixture thereof.

The pet food compositions disclosed herein may be wet or dry compositions, and the ingredients can be either incorporated into the food composition and/or on the surface of any composition component, such as, for example, by spraying, agglomerating, dusting, or precipitating on the surface. Additionally, the pet food compositions may be formulated and produced to be in various forms and/or consistencies. For instance, the pet food compositions may, for example, be a dry, moist or semi-moist animal food composition. “Semi-moist” refers to a food composition containing from about 25 to about 35% moisture. “Moist” food refers to a food composition that has a moisture content of about 60 to 90% or greater. “Dry” food refers to a food composition with about 3 to about 12% moisture content and is often manufactured in the form of small bits or kibbles.

The food products may also include components of more than one consistency, for example, soft, chewy meat-like particles or pieces as well as kibble having an outer coating and an inner “core” component. In some embodiments, the pet food compositions may be in the form of a kibble or food kibble. As used herein, the term “kibble” or “food kibble” refers to a particulate pellet, e.g., like a component of feline or canine feeds. In some embodiments, a food kibble has a moisture, or water, content of less than 15% by weight. Food kibbles may range in texture from hard to soft. Food kibbles may range in internal structure from expanded to dense. Food kibbles may be formed by an extrusion process or a baking process. In non-limiting examples, a food kibble may have a uniform internal structure or a varied internal structure. For example, a food kibble may include a core and a coating to form a coated kibble. It should be understood that when the term “kibble” or “food kibble” is used, it can refer to an uncoated kibble or a coated kibble.

The composition of the present disclosure can additionally comprise other additives in amounts and combinations familiar to one of skill in the art. Such additives should be present in amounts that do not impair the purpose and effect provided by the invention. Examples of additives include substances with a stabilizing effect, organoleptic substances, processing aids, and substances that provide nutritional benefits.

Stabilizing substances may include, by way of example, substances that tend to increase the shelf life of the pet food composition. Other examples of other such additives potentially suitable for inclusion in the compositions of the invention include, for example, preservatives, antioxidants, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. Additives for coloring, palatability, and nutritional purposes can include colorants, salts (including, but not limited to, sodium chloride, potassium citrate, potassium chloride, and other edible salts), vitamins, minerals, and flavoring. Other additives can include glucosamine, chondroitin sulfate, vegetable extracts, herbal extracts, etc.

The concentration of such additives in the pet food composition typically can be up to about wt. %, based on the total weight of the pet food composition on a dry matter basis. For example, the additives may be present in an amount from about 0.01 to about 5 wt. %, about 0.01 to about 4 wt. %, about 0.01 to about 4 wt. %, about 0.01 to about 3 wt. %, about 0.01 to about 2 wt. %, about to about 1 wt. %; about 0.1 to about 5 wt. %, about 0.1 to about 4 wt. %, about 0.1 to about 4 wt. %, about 0.1 to about 3 wt. %, about 0.1 to about 2 wt. %, about 0.1 to about 1 wt. %; about 1 to about 5 wt. %, about 1 to about 4 wt. %, about 1 to about 4 wt. %, about 1 to about 3 wt. %, about 1 to about 2 wt. %; about 2 to about 5 wt. %, about 2 to about 4 wt. %, about 2 to about 4 wt. %, about 2 to about 3 wt. %; about 3 to about 5 wt. %, about 3 to about 4 wt. %; or about 4 to about 5 wt. %, based on the total weight of the pet food composition on a dry matter basis. In some embodiments, the concentration of such additives (particularly where such additives are primarily nutritional balancing agents, such as vitamins and minerals) is from about 0 to about 2.0% by weight, based on the total weight of the pet food composition on a dry matter basis. The amount of additives comprising vitamins may be in addition to the amount of vitamin B discussed above. In some embodiments, the concentration of such additives (again, particularly where such additives are primarily nutritional balancing agents) is from about 0 to about 1.0% by weight, based on the total weight of the pet food composition on a dry matter basis. Although the list of foregoing additives may be potentially suitable in some embodiments, one or more of the foregoing additives may be excluded from other embodiments of the pet food composition.

In specific embodiments, the pet food compositions and food products are formulated to address specific nutritional differences between species and breeds of animals, as well as one of more of the attributes of the animal. For example, cat foods, for example, are typically formulated based upon the life stage, age, size, weight, body composition, and breed.

Sources of proteins, carbohydrates, fats, vitamins, minerals, balancing agents, and the like, suitable for inclusion in the pet food compositions, and particularly in the food products to be administered in methods provided herein, may be selected from among those conventional materials known to those of ordinary skill in the art.

The pet food compositions may include, or in some instance exclude, one or more ingredients selected from beef broth, brewers dried yeast, egg, egg product, flax meal, DL methionine, amino acids, leucine, lysine, arginine, cysteine, cystine, aspartic acid, polyphosphates, sodium pyrophosphate, sodium tripolyphosphate; zinc chloride, copper gluconate, stannous chloride, stannous fluoride, sodium fluoride, triclosan, glucosamine hydrochloride, chondroitin sulfate, green lipped mussel, blue lipped mussel, methyl sulfonyl methane (MSM), boron, boric acid, phytoestrogens, phytoandrogens, genistein, diadzein, Lcarnitine, chromium picolinate, chromium tripicolinate, chromium nicotinate, acid/base modifiers, potassium citrate, potassium chloride, calcium carbonate, calcium chloride, sodium bisulfate; eucalyptus, lavender, peppermint, plasticizers, colorants, flavorants, sweeteners, buffering agents, slip aids, carriers, pH adjusting agents, natural ingredients, stabilizers, biological additives such as enzymes (including proteases and lipases), chemical additives, coolants, chelants, denaturants, drug astringents, emulsifiers, external analgesics, fragrance compounds, humectants, opacifying agents (such as zinc oxide and titanium dioxide), antifoaming agents (such as silicone), preservatives (such as butylated hydroxytoluene (BHT) and butylated hydroxyanisole (BHA), propyl gallate, benzalkonium chloride, EDTA, benzyl alcohol, potassium sorbate, parabens and mixtures thereof), reducing agents, solvents, hydrotropes, solubilizing agents, suspending agents (non-surfactant), solvents, viscosity increasing agents (aqueous and non-aqueous), sequestrants, keratolytics, egg shell membrane, ancient grain, and a combination of two or more thereof.

The pet food composition may be produced by various methods to achieve the desired pet food composition or desired form for the pet food composition. For example, dry food may be baked or extruded, then cut into individual shaped portions, such as kibbles. In some embodiments, the pet food composition may be prepared in a canned or wet form using conventional food preparation processes known to those of ordinary skill in the art. Typically, ground animal proteinaceous tissues are mixed with the other ingredients, such as cereal grains, suitable carbohydrate sources, fats, oils, and balancing ingredients, including special purpose additives such as vitamin and mineral mixtures, inorganic salts, cellulose, beet pulp and the like, and water in an amount sufficient for processing. The ingredients are mixed in a vessel suitable for heating while blending the components. Heating the mixture is carried out using any suitable manner, for example, direct steam injection or using a vessel fitted with a heat exchanger. Following addition of all of the ingredients of the formulation, the mixture may be heated to a temperature of from 50° F. to 212° F. Although temperatures outside this range can be used, they may be commercially-impractical without the use of other processing aids. When heated to the appropriate temperature, the material will typically be in the form of thick liquid, which is dispensed into cans. A lid is applied and the container is hermetically sealed. The sealed can is then placed in convention equipment designed for sterilization of the contents. Sterilization is usually accomplished by heating to temperatures of greater than 230° C. for an appropriate time depending on the temperature used, the nature of the composition, and related factors. The pet food compositions and food products of the present disclosure can also be added to or combined with food compositions before, during, or after their preparation.

In some embodiments, the food products may be prepared in a dry form using convention processes known to those of ordinary skill in the art. Typically, dry ingredients, including dried animal protein, plant protein, grains and the like are ground and mixed together. Liquid or moist ingredients, including fats, oils water, animal protein, water, and the like are added combined with the dry materials. The specific formulation, order of addition, combination, and methods and equipment used to combine the various ingredients can be selected from those known in the art. For example, in certain embodiments, the resulting mixture is process into kibbles or similar dry pieces, which are formed using an extrusion process in which the mixture of dry and wet ingredients is subjected to mechanical work at high pressure and temperature, forced through small openings or apertures, and cut off into the kibbles, e.g., with a rotating knife. The resulting kibble can be dried and optionally coated with one or more topical coatings comprising, e.g., flavors, fats, oils, powdered ingredients, and the like. Kibbles may also be prepared from dough by baking, rather than extrusion, in which the dough is placed into a mold before dry-heat processing.

In preparing a composition, any ingredient generally may be incorporated into the composition during the processing of the formulation, e.g., during and/or after mixing of the other components of the composition. Distribution of these components into the composition can be accomplished by conventional means. In certain embodiments, ground animal and/or poultry proteinaceous tissues are mixed with other ingredients, including nutritional balancing agents, inorganic salts, and may further include cellulose, beet pulp, bulking agents and the like, along with sufficient water for processing.

In accordance with another aspect, provided is a method for reducing tarter for a pet, the method comprising feeding a pet a pet food composition disclosed herein. The method may include providing and/or feeding the canine a pet food composition disclosed herein. In some instances, the method may include providing and/or feeding the canine the pet food compositions for 1 or more days, preferably 5 or more days, preferably 7 or more days, preferably 10 or more days, preferably 14 or more days, preferably 30 or more days, or preferably 42 or more days. The method may include feeding the pet one time a day, two times a day, three times a day, or in some embodiments four or more times a day.

According to another aspect, provided is a method for alleviating, mitigating, and/or reducing several indications or conditions, including, but not limited to, cancer, inflammatory bowel disease, diabetes, obesity, hyperglycemia, hypertriglyceridemia, chronic inflammation, antibiotic resistance of the gut microbiome, or pathogenic dysbiosis of the gut microbiome, neurological seizures, high blood glucose, metabolic stress, or combinations thereof. The method typically comprising administering or feeding the pet a pet food composition disclosed herein, e.g., as discussed above. The administration of the pet food comprising the dietary composition shifts the animal's metabolism from glucose-converting metabolism to fat-burning metabolism.

### EXAMPLES

### Example 1

Two non-limiting, exemplary pet food compositions (Example Compositions A and B) were prepared in accordance with aspects of the invention. The formulation for Example Compositions A and B are provided in Table 1, while the macronutrients of Examples A and B are provided in Table 2. Two comparative compositions (Composition 1 and Composition 2) was evaluated for comparison purposes. Composition 1 was a negative control and was not formulated to provide a chemical or mechanical benefit. Composition 2 was a positive control and was formulated to provide a mechanical benefit, but not a chemical benefit. The formulation and macronutrients of comparative Composition 1 and positive control Composition 2 are provided in Tables 1 and 2, respectively.

A study of 20 adult canines was conducted to evaluate the effect of Examples A and B in comparison to two comparative food compositions (Comparative Compositions 1 and 2). All of the canines had ages ranging from 4.2 years to 7.6 years and body weights ranging from 12.33 to 22.13 kg. Additionally, the canines were selected from male and female dogs regardless of their reproductive status.

The 20 adult canines were divided into four groups of 5 dogs, based on their gender, age and their whole mouth plaque scores, which was measured before the canines received any of the pet food compositions pursuant to the study. Each group of canines was then fed one of four possible diets for 28 days. At the end of the 28 days, the canines were evaluated. Each of the groups subsequently received a different pet food composition for 28 days, and were then evaluated. This procedure was repeated until each group was fed each pet food composition for 28 days.

The ingredients were obtained from commercial sources and formulated according to AAFCO and NRC nutrition recommendations.

TABLE 1

| Ingredients (%) | Comp. 1 | Comp. 2 | Ex. A | Ex. B |
|---|---|---|---|---|
| Antioxidant, Verdilox | 0.04 | | 0.05 | 0.05 |
| Oat Fiber, Fruit, Vegetable blend | 0.04 | | | |
| Mineral premix | 0.06 | 0.07 | 0.125 | 0.125 |
| Taurine | 0.06 | 0.07 | 0.25 | 0.25 |
| Vitamin E adsorbate (50%) | 0.09 | | | |
| Vitamin E oil, (29%) | | 0.17 | 0.25 | 0.25 |
| Vitamin Premix | 0.2 | 0.13 | 0.2 | 0.2 |
| Sodium chloride, iodized | 0.41 | 0.29 | | 0.4 |
| Choline chloride (70%) | 0.44 | 0.2 | 0.4 | 0.4 |
| Lactic acid (84%) | 1.2 | 1.2 | 1.5 | 1.5 |
| Natural flavor, Pork, Liver, Digest, D′T | 1.25 | | | |
| Soybean oil | 2.5 | 1 | | |
| Chicken Meal, and/or Chicken liver digest | 5.6 | 1 | | |
| Rice, brewers | 3 | 30.5 | | |
| Soybean, meal | 4.8 | | | |
| Corn, yellow, whole | 11.6 | 23.5 | | |
| Barley, pearled, cracked | 11.6 | | | |
| Wheat, red, whole | 11.6 | | | |
| Sorghum, whole | 11.6 | | | |
| Corn, gluten, meal | 11.6 | | | |
| Chicken, ground, fresh | 14 | | | |
| Pork Fat (Choice White Grease) or | 4.3 | 9.5 | 20 | 15.1 |
| Chicken Fat Refined | | | | |
| Poultry By-Product Meal, Low Ash | | 14.6 | | |
| Eggs (dried), Pork meat protein | | | 49 | 29 |
| isolate, and Chicken (dried) | | | | |
| Gelatin Colloid | | | 5 | 2 |
| Palatant, | | | 4 | 5 |
| Calcium carbonate or Calcium sulfate | 0.72 | 0.52 | 1.5 | 0.5 |
| Carnitine, 1, 10% | | | | 0.75 |
| Methionine, Lysine (1, hydrochloride) and/or | | 0.3 | 0.6 | 0.75 |
| L-Cysteine hydrochloride monohydrate, and/or | | | | |
| Betaine base anhydrous, trimethylglycine | | | | |
| Soybean, millrun, ground | | 6.2 | | |
| C8 medium chain triglyceride | | | | 8 |
| Hydrolyzed whey protein, and Hydrolyzed casein | | | | 6 |
| Flax seed (whole brown) and/or Oat bran | 0.58 | | 5 | 7.5 |
| Beet pulp, Citrus pulp, Pectin, and Apple Pomace | 2.5 | | | 4.5 |
| Cellulose and/or Methyl Cellulose | | 10 | 5 | 4.5 |
| Pecan shells | | | 3 | 5 |
| Psyllium husks, (95%) | | | 0.75 | 1 |
| Fish oil | | | | 1 |
| Lipoic acid, alpha (5%) | | | | 0.5 |
| Cranberry, pressed | | | | 0.5 |
| Potato Starch | | | | 2 |
| Dicalcium phosphate, Sodium tripolyphosphate, | 0.5 | 0.7 | 1.5 | 2.5 |
| Magnesium oxide, Potassium alginate, | | | | |
| Potassium citrate, and/orPotassium chloride | | | | |

TABLE 2

| | Comp. 1 | Comp. 2 | Ex. A | Ex. B |
|---|---|---|---|---|
| Macronutrients | (wt. %) | (wt. %) | (wt. %) | (wt. %) |
| Moisture | 8 | 8.5 | 6.7 | 8.2 |
| Protein | 21.9 | 15.3 | 45.3 | 32.3 |
| Fat | 13.5 | 14.8 | 32.1 | 33.4 |
| Crude Fiber | 1.9 | 9.7 | 4.6 | 5.4 |
| Ash | 5 | 3.9 | 6.7 | 5.8 |
| Dietary Fiber | 8.9 | 16.6 | 11.1 | 14.4 |
| Soluble Fiber | 2 | 0.9 | 0.6 | 2.2 |
| Insoluble Fiber | 6.7 | 15.2 | 10.6 | 12.2 |
| Nitrogen Free Extract (NFE) | 49.6 | 47.7 | 4.8 | 14.9 |

At the start and end of each diet phase, a full mouth dental prophylaxis and dental examination was conducted. Plaque and calculus accumulation was quantified using the Logan/Boyce method. Gingivitis scores were calculated using the following metrics: 0=normal; 0.5=mild inflammation with slight redness; 1=moderate inflammation and redness, but no bleeding on probing; 2=moderate inflammation with severe redness and bleeding on proving; and 3=severe inflammation and redness, edema, ulceration, and spontaneous bleeding.

The foregoing findings support the conclusion that a low carbohydrate pet food composition significantly reduces the levels of tartar formation, which may lead to improved oral health in dogs. Example Composition A provided a noticeable reduction in mouth tartar as compared to Comparative Composition 1, while Example Composition B provided a significant reduction in mouth tartar as compared to Comparative Composition 1.

TABLE 3

| | | | | Comp, 2 | | |
|---|---|---|---|---|---|---|
| Whole Mouth | Food Effect | | Comp. 1 | (Positive | | |
| Scoring | (P value) | Baseline | (Comparative) | Control) | Ex. A | Ex. B |
| Plaque | <0.0001 | 5.05 ± 0.35^{A} | 4.61 ± 0.31^{A} | 3.51 ± 0.26^{B} | 4.73 ± 0.23^{A} | 4.53 ± 0.29^{A} |
| Tartar | <0.0001 | 1.93 ± .022^{A} | 1.35 ± 0.16^{B} | 0.72 ± 0.11^{D} | 1.07 ± 0.13^{BC} | 1.03 ± 0.14^{C} |
| Gingivitis | <0.0001 | 0.88 ± 0.04^{A} | 0.68 ± 0.06^{B} | 0.54 ± 0.06^{C} | 0.72 ± 0.06^{B} | 0.72 ± 0.06^{B} |

### Example 1

Sub-gingival plaque samples was also obtained from the canines of Example 1. Specifically, for each canine, sub-gingival plaque sample was collected from healthy tooth and unhealthy tooth. Healthy and unhealthy tooth was confirmed based on the tooth plaque, tartar and gingivitis scores. If the subject did not have unhealthy tooth then sub-gingival plaque samples was collected from two different healthy teeth. If the subject do not have healthy tooth then sub-gingival plaque samples was collected from two different unhealthy teeth.

The sub-gingival plaque samples were analyzed for microbiome sequencing by extracting total DNA from frozen samples by using Qiagen DNeasy Power Biofilm extraction kit. Following total DNA extraction, 16s rDNA amplicon was developed from the samples by employing PCR using the primer sets spanning the V1 and V3 hypervariable regions and the amplicons were subsequently analyzed qualitatively by an Agilent 2100 Bioanalyzer. Index PCR was then performed, followed by library quantification and normalization.

For the microbiome analyses, the abundance data at the phyla, family, and genera levels, were first filtered based upon by prevalence in the data set and the percentage of reads retained per sample. The data was then filtered and statistical analysis was performed.

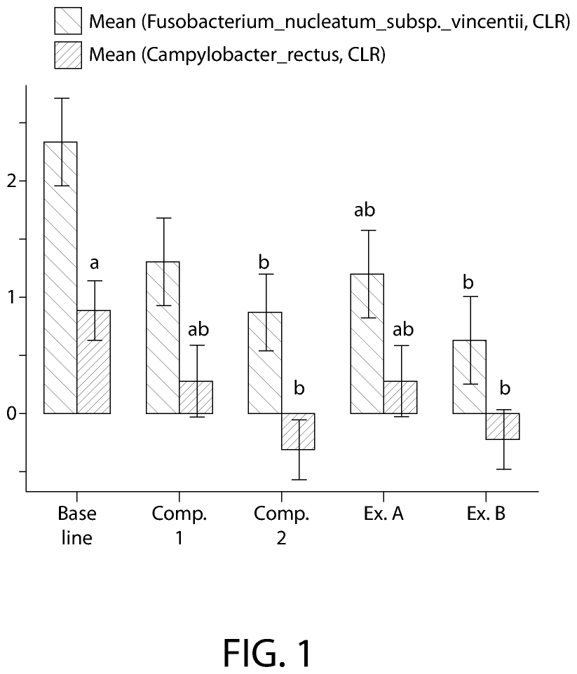

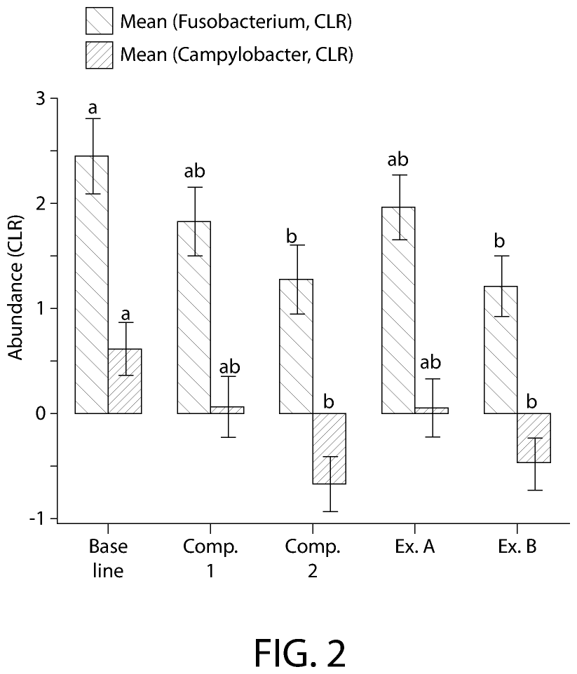

Based on the sub-gingival plaque microbiome analyses, it was surprisingly determined that bacterial genera/species belonging to “Orange complex” was significantly reduced (see FIGS. 1 and 2) for the canniness fed Example Composition B compared to Comparative Composition 1 and Example Composition A. Specifically, the abundance levels of these bacterial genera/species are equivalent to the dogs fed with Comparative Composition 2 (positive control, mechanical effect), which was proven to reduce whole mouth plaque and tartar scores.

Orange complex consists of three species: Fusobacterium nucleatum, Campylobacter rectus and Prevotella intermedia. Orange complex species may be considered as bridging species to establish “Red complex” species in the process of plaque (biofilm) maturation. “Red complex” species may be considered as potential pathobiont to trigger gum inflammation, leading to gingivitis and irreversible periodontitis condition. Fusobacterium nucleatum, a member of “Orange complex” is a common member of the oral microbiota in both periodontal health and disease and metabolically integrates beneficial (commensals) bacterial species considered as early colonizers with potential pathobiont (red complex) in the plaque maturation process. Besides causing gingivitis and periodontitis, it is believed that Fusobacterium nucleatum also plays a role in halitosis, dental pulp infection, and potentially oral cancer. Halitosis is a common condition characterized by an unpleasant and disgusting odor (bad breath) emanating from the mouth, also common both in human and companion animals.

A significant reduction of Campylobacter rectus in the dogs fed Example Composition as compared to Comparative Composition 1 and Example Composition A was also observed. Campylobacter rectus has the potential to shift the oral microbial community in a positive way by reducing “Red complex” species critical for initiation and progression of periodontitis. Overall, Example Composition B provided a significant reduction in the levels of “Orange complex” species, such as Fusobacterium nucleatum and Campylobacter rectus, which are considered as bridging species and play a critical role in the plaque maturation process.

1-51. (canceled)

52. A pet food composition comprising:
about 20 wt. % or more of fat comprising one or more medium chain triglyceride, wherein at least one medium chain triglyceride comprises an octanoate component, the at least one medium chain triglyceride comprising the octanoate component comprising about wt. % or more of the fat, based on the total weight of the fat on a dry matter basis;
a protein; and
a fiber;
wherein the pet food composition comprises about 10 wt. % or less of carbohydrate and about 5 wt. % or less of digestible starch,
wherein the pet food composition has a ketogenic ratio of about 1.5 or greater as calculated from Formula 1: (0.9 F+0.46 P)/(0.1 F+0.58 P+C), wherein F is the weight percentage of fat, P is the weight percentage of protein, and C is the weight percentage of carbohydrate, and all weight percentages are based on the total weight of the pet food composition on a dry matter basis.

53. The pet food composition according to claim 52, wherein the pet food has ketogenic ratio is about 2 or greater as calculated from Formula 1: (0.9 F+0.46 P)/(0.1 F+0.58 P+C).

54. The pet food composition according to claim 52, wherein the one or more medium chain triglyceride further comprises at least one additional medium chain triglyceride having an aliphatic chain component selected from pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, and mixtures thereof.

55. The pet food composition according to claim 52, wherein the pet food composition has a total amount of medium chain triglycerides having a decanoate component of about 1 wt. % or less, based on the total weight of fat in the pet food composition on a dry matter basis.

56. The pet food composition according to claim 52, wherein pet food composition has a weight ratio of the medium chain triglyceride having the aliphatic chain comprising the octanoate component to a medium chain triglyceride having the aliphatic chain comprising the decanoate component of from about 200:1 to about 500:1.

57. The pet food composition according to claim 52 further comprising one or more fatty acid selected from omega-3 fatty acids, omega-6 fatty acids, lauric acid, myristic acid, palmitic acid, palmitoleic acid, margaric acid, margaroleic acid, stearic acid, oleic acid, stearidonic acid, gadoleic acid, behenic acid, erucic acid, docosatetra acid, and a combination of two or more thereof.

58. The pet food composition according to claim 57, comprising an omega-3 fatty acid selected from linolenic acid, stearidonic acid, eicosatetraenoic acid, eicosapentaenoic acid, docosapentaenoic acid, docosahexaenoic acid, and a combination of two or more thereof.

59. The pet food composition according to claim 57, comprising an omega-6 fatty acid selected from linoleic acid, calendic acid, eicosadienoic acid, arachidonic acid, docosadienoic acid, adrenic acid, osbond acid, tetracosatetraenoic acid, tetracosapentaenoic acid, and a combination of two or more thereof.

60. The pet food composition according to claim 57, wherein the pet food composition has a weight ratio of omega-3 fatty acids to omega-6 fatty acids of from about 0.5:1 to about 7:1.

61. The pet food composition according to claim 52, further comprising at least one phenol selected from flavonoids, lignans, metabolic enhancers, and a combination of two or more thereof.

62. The pet food composition according to claim 61, wherein the lignin is selected from coniferyl alcohol, sinapyl alcohol, and paracoumaryl alcohol.

63. The pet food composition according to claim 61, wherein the at least one metabolic enhancer is selected from lipoic acid, taurine, carnitine, betaine (trimethylglycine), coenzyme Q10, beta-alanine, pyrroloquinoline quinone (PQQ), creatine, and a combination of two or more thereof.

64. The pet food composition according to claim 63, wherein the at least one metabolic enhancer is lipoic acid or carnitine.

65. A pet food composition comprising:
from about 20 to about 60 wt. % of fat comprising one or more medium chain triglyceride, wherein at least one medium chain triglyceride comprises an octanoate component, wherein the at least one medium chain triglyceride comprising the octanoate component comprises about 10 wt. % or more of the fat, based on the total weight of the fat on a dry matter basis;
from about 15 to about 55 wt. % of protein;
from about 10 to about 60 wt. % of a fiber; and
one or more fatty acid,
wherein the pet food composition comprises about 10 wt. % or less of carbohydrate and about 5 wt. % or less of digestible starch, and wherein all weight percentages are based on the total weight of the pet food composition on a dry matter basis.

66. The pet food composition according to claim 65, wherein the pet food composition has a ketogenic ratio of about 1.5 or greater as calculated from Formula 1: (0.9 F+0.46 P)/(0.1 F+0.58 P+C), wherein F is the weight percentage of fat, P is the weight percentage of protein, and C is the weight percentage of carbohydrate.

67. The pet food composition according to claim 65, wherein the pet food composition contains less than about 2 wt. % of phosphorus.

68. The pet food composition according to claim 65, wherein the fiber is at least partially comprised of pecan shell, almonds shell, walnut shell, citrus pulp, beet pulp, pear skin, or a combination of two or more thereof.

69. The pet food composition according to claim 68, wherein the fiber is at least partially comprised of almonds shell, walnut shell, pecan shell, or a combination of two or more thereof.

70. The pet food composition according claim 65, further comprising one or more sclereid.

71. The pet food composition according to claim 70, wherein the one or more sclereid comprises at least one polyphenol.
